SEQUENCE LISTING (6 sequences)

SEQ ID NO: 1 - protein, 15 aa, Artificial Sequence
  Synthetic
MFVFLVLLPLVSSQC

SEQ ID NO: 2 - protein, 15 aa, Artificial Sequence
  Synthetic
MKIILFLALITLATC

SEQ ID NO: 3 - protein, 15 aa, Artificial Sequence
  Synthetic
MKFLVFLGIITTVAA

SEQ ID NO: 4 - protein, 19 aa, Artificial Sequence
  Synthetic
  MISC_FEATURE (16)..(16): X is K, D, H, or E
  MISC_FEATURE (17)..(19): X is K, D, H, E, or absent
MFVFLVLLPLVSSQCXXXX

SEQ ID NO: 5 - protein, 19 aa, Artificial Sequence
  Syhnthetic
  MISC_FEATURE (16)..(16): X is K, D, H, or E
  MISC_FEATURE (17)..(19): X is K, D, H, E, or absent
MKIILFLALITLATCXXXX

SEQ ID NO: 6 - protein, 19 aa, Artificial Sequence
  Synthetic
  MISC_FEATURE (16)..(16): X is K, D, H, or E
  MISC_FEATURE (17)..(19): X is K, D, H, E, or absent
MKFLVFLGIITTVAAXXXX

(19) United States
(12) Patent Application Publication
(10) Pub. No.: US 2023/0119806 A1
(43) Pub. Date: Apr. 20, 2023

(54) ANTIGEN SPECIFIC EPITOPE BASED ANTI-INFECTIVE VACCINES

(71) Applicant: VAXIL BIOTHERAPEUTICS LTD., Nes Ziona (IL)

(72) Inventors: David GOREN, Tel Aviv (IL); Mordechai APPLEBAUM, Jerusalem (IL); Ron PINKUS, Raanana (IL)

(21) Appl. No.: 17/910,085

(22) PCT Filed: Mar. 9, 2021
(86) PCT No.: PCT/IL2021/050262

§ 371 Date: Sep. 8, 2022

Related U.S. Application Data
(60) Provisional application No. 62/987,310, filed on Mar. 9, 2020.
(60) Provisional application No. 63/000,213, filed on Mar. 26, 2020.

(51) Int. Cl. A61K 39/215; A61K 45/06; A61P 31/14; C07K 14/005

(52) U.S. Cl. CPC A61K 39/215; A61K 45/06; A61P 31/14; C07K 14/005; C12N 2770/20071; C12N 2770/20022; C12N 2770/20034; A61K 2039/572

(57) ABSTRACT

The present invention relates to an immunogenic composition directed against intracellular pathogens, as well as to peptide vaccines including the immunogenic composition and methods for treating or preventing infections caused by intracellular pathogens.

### CROSS-REFERENCE TO RELATED APPLICATIONS

This application claims the benefit of priority to U.S. Provisional Patent Application No. 62/987,310, titled: “ANTIGEN SPECIFIC EPITOPE-BASED ANTI-INFECTIVE VACCINES”, filed, Mar. 9, 2020, and U.S. Provisional Patent Application No. 63/000,213, titled: “ANTIGEN SPECIFIC EPITOPE—BASED ANTI-INFECTIVE VACCINES”, filed, Mar. 26, 2020, the contents of which are incorporated herein by reference in their entirety.

### FIELD OF THE INVENTION

The present invention relates to immunogenic compositions directed against intracellular pathogens, as well as to peptide vaccines comprising the immunogenic compositions and methods for treating or preventing infections caused by intracellular pathogens.

### BACKGROUND

SARS-CoV-2 is the virus that causes COVID-19, a type of coronavirus. A subgroup of Coronaviruses is a group of viruses that cause respiratory tract infections in humans. They are enveloped viruses with a positive-sense single-stranded RNA genome. There are yet to be vaccines or antiviral drugs to prevent or treat human coronavirus infections. COVID-19 has become a major public health issue with the 2019-2020 global outbreak as it is a novel coronavirus with efficient human-to-human transmission. Those infected may be either asymptomatic or develop symptoms including fever, cough and shortness of breath. Cases can progress to pneumonia, multi-organ failure, and death in the most vulnerable. An effective vaccine is essential in order to control the spread of COVID-19.

SUMMARY

According to a first aspect, there is provided an immunogenic composition, comprising at least one peptide selected from the group consisting of: MFVFVFLVLLPLVSSQC (SEQ ID NO: 1), MKIILFLALITLATC (SEQ ID NO: 2) and MKFLVFLGIITTVAA (SEQ ID NO: 3); and optionally at least one adjuvant.

According to another embodiment, the at least one peptide is a peptide consisting of an amino acid sequence selected from the group listed in Table 1 (SEQ ID NOs: 1-3).

According to another embodiment, the peptide has an amino acid sequence having at least 85% homology with said amino acid sequence selected from the group listed in Table 1 (SEQ ID NOs: 1-3). According to another embodiment, the peptide has an amino acid sequence having at least 90% homology with said amino acid sequence selected from the group listed in Table 1 (SEQ ID NOs: 1-3). According to another embodiment, the peptide has an amino acid sequence having at least 95% homology with said amino acid sequence selected from the group listed in Table 1 (SEQ ID NOs: 1-3). According to another embodiment, the one or more of said at least one peptides has a C-terminal amide.

According to another embodiment, the immunogenic composition exhibits, upon administration, a combined activation of both CD4+ and CD8+ T cells.

According to another embodiment, the immunogenic composition is configured (e.g., formulated) for co-administration with one or more anti-infective agents.

According to another aspect, there is provided a vaccine comprising the immunogenic composition of the invention.

According to another aspect, there is provided an isolated antigen-presenting cell preloaded with the peptide according to the present invention.

According to other aspect, there is provided a method of treating or preventing a pathogenic infection comprising administering the immunogenic composition of the present invention to a subject in need thereof

According to another aspect, there is provided a method of treating or preventing a pathogenic infection comprising administering an enriched T cell population to a subject in need thereof, wherein said enriched T cell population is obtained by administering the immunogenic composition according to the present invention, to a T cell population in vitro.

According to another embodiment, the pathogenic infection is a coronavirus. According to another embodiment, the coronavirus is COVID-19.

According to another aspect, there is provided a method for generating anti-coronavirus antibodies comprising administering the immunogenic composition of the invention to an animal or a human subject.

According to another embodiment, the anti-coronavirus antibodies are antibodies against SARS-CoV-2 signal peptides.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

FIG. 1 includes a graph showing enzyme-linked immunosorbent assay (ELISA) of sera obtained from BALB/c mice immunized with signal peptides (SPs). Sera of BALB/c mice were obtained 10 days post the last immunization. Values were normalized to control.

FIG. 2 includes a graph showing ELISA of sera obtained from C57BL/6 mice immunized with SPs. Sera of the C57BL/6 mice were obtained 20 days post the last immunization. Values were normalized to control.

FIG. 3 includes a graph showing an ELISpot assay of interleukin 2 following a SP stimulation experiment on splenocytes of immunized C57BL/6 mice.

FIG. 4 includes a graph showing an ELISpot assay of interferon gamma following a SP stimulation experiment on splenocytes of immunized BALB/c mice.

### DETAILED DESCRIPTION

The present invention is directed to immunogenic compositions comprising at least one peptide selected from the group consisting of SEQ ID NOs: 1-3 and an optional pharmaceutically acceptable carrier and/or adjuvant. The present invention is also directed to vaccines made from the compositions. The present invention further concerns methods of treating and preventing infections and methods of generating antibodies.

By a first aspect, there is provided an immunogenic composition comprising at least one peptide selected from the group consisting of MFVFLVLLPLVSSQC (SEQ ID NO: 1), MKIILFLALITLATC (SEQ ID NO: 2) and MKFLVFLGIITTVAA (SEQ ID NO: 3) and optionally at least one adjuvant.

In some embodiments, the at least one peptide further comprises at least one charged amino acid residues in any one of the C-Terminus, N-terminus, or both ends of the peptide. In some embodiments, the at least one peptide further comprises at least one charged amino acid residues in the C-Terminal end of the peptide.

In some embodiments, the at least one charged amino acids is at least two charged amino acids. In some embodiments, the at least one charged amino acids is at least three charged amino acids. In some embodiments, the at least one charged amino acids is at least four charged amino acids.

In some embodiments, the at least one charged amino acids is, independently, selected from the group consisting of Lysine (“K”), Aspartic acid (“D”), Glutamic acid (“E”), and Histidine (“H”), and any combination thereof. In some embodiments, the at least one charged amino acids is KDHE.

In some embodiments, the at least one peptide comprises or consists of the amino acid sequence as set forth in MFVFLVLLPLVSSQCX₁X₂X₃X₄ wherein X₁ is any one of K, D, H, E; X₂ is any one of K, D, H, E or absent; X₃ is any one of K, D, H, E or absent; and X₄ is any one of K, D, H, E or absent (SEQ ID NO: 4). In one embodiment, X₁X₂X₃X₄is KDHE.

In some embodiments, the at least one peptide comprises or consists of the amino acid sequence as set forth in MKIILFLALITLATCX₁X₂X₃X₄ wherein X₁ is any one of K, D, H, E; X₂ is any one of K, D, H, E or absent; X₃ is any one of K, D, H, E or absent; and X₄ is any one of K, D, H, E or absent (SEQ ID NO: 5). In one embodiment, X₁X₂X₃X₄is KDHE.

In some embodiments, the at least one peptide comprises or consists of the amino acid sequence as set forth in MKFLVFLGIITTVAAX₁X₂X₃X₄ wherein X₁ is any one of K, D, H, E; X₂ is any one of K, D, H, E or absent; X₃ is any one of K, D, H, E or absent; and X₄ is any one of K, D, H, E or absent (SEQ ID NO: 6). In one embodiment, X₁X₂X₃X₄is KDHE.

In some embodiments, the addition of at least one charged amino acids improves (e.g., reduces) hydrophobicity of the peptide, as compared to the same peptide devoid of the charged-amino acid addition (and, therefore, may reduce aggregation of the peptide).

In some embodiments, the addition of at least one charged amino acids improves the stability of the peptide, as compared to the same peptide devoid of the charged-amino acid addition.

In some embodiments, the addition of at least one charged amino acids improves the serum half-life of the peptide, as compared to the same peptide devoid of the charged-amino acid addition.

In some embodiments, the addition of at least one charged amino acids improves the solubility of the peptide (e.g., increases compatibility for intradermal or intramuscular formulations), as compared to the same peptide devoid of the charged-amino acid addition.

In some embodiments, the addition of at least one charged amino acids increases immunogenicity of the peptide, as compared to the same peptide devoid of the charged-amino acid addition.

In some embodiments, the peptide consists of an amino acid sequence selected from the group consisting of SEQ ID NOs: 1-3. In some embodiments, the immunogenic composition comprises SEQ ID NO 1. In some embodiments, the immunogenic composition comprises SEQ ID NO 2. In some embodiments, the immunogenic composition comprises SEQ ID NO 3. In some embodiments, the immunogenic composition comprises two or more peptides.

In some embodiments, the peptide consists of an amino acid sequence selected from the group consisting of SEQ ID NOs: 4-6. In some embodiments, the immunogenic composition comprises SEQ ID NO: 4. In some embodiments, the immunogenic composition comprises SEQ ID NO: 5. In some embodiments, the immunogenic composition comprises SEQ ID NO: 6. In some embodiments, the immunogenic composition comprises two or more peptides.

In some embodiments, the peptide is substantially homologous to the sequences listed in SEQ ID NOs 1-3, with the addition of a fragment or addition that does not substantially reduce the peptide's activity. In some embodiments, the peptide is substantially homologous to the sequences listed in SEQ ID NOs 4-6, with the addition of a fragment or addition that does not substantially reduce the peptide's activity.

In some embodiments, the peptide has an amino acid sequence having at least 85% homology with the amino acid sequence listed as SEQ ID NO: 1. In some embodiments, the peptide has an amino acid sequence having at least 75%, at least 80%, at least 85%, at least 90%, at least 95% homology to the amino acid sequence listed as SEQ ID NO 1. Each possibility represents a separate embodiment of the invention.

In some embodiments, the peptide has an amino acid sequence having at least 85% homology with the amino acid sequence listed as SEQ ID NO 2. In some embodiments, the peptide has an amino acid sequence having at least 75%, at least 80%, at least 85%, at least 90%, at least 95% homology to the amino acid sequence listed as SEQ ID NO 2. Each possibility represents a separate embodiment of the invention.

In some embodiments, the peptide has an amino acid sequence having at least 85% homology with the amino acid sequence listed as SEQ ID NO 3. In some embodiments, the peptide has an amino acid sequence having at least 75%, at least 80%, at least 85%, at least 90%, at least 95% homology to the amino acid sequence listed as SEQ ID NO 3. Each possibility represents a separate embodiment of the invention.

### Immunogenic Composition

The term “immunogenic composition” as used herein refers to a composition that is able to produce an immune response.

In some embodiments, the immunogenic composition exhibits, upon administration, activation of T cells. In some embodiments, the immunogenic composition exhibits, upon administration, activation of CD4+ T cells. In some embodiments, the immunogenic composition exhibits, upon administration, activation of CD8+ T cells. In some embodiments, the immunogenic composition exhibits, upon administration, combined activation of CD4+ and CD8+ T cells.

In some embodiments, the immunogenic composition exhibits, upon administration, production of specific antibodies (of any immunoglobin class) against epitopes within the said peptides. Each possibility represents a separate embodiment.

As used herein, the nomenclature used to describe peptides of the invention follows the conventional practice wherein the amino group (N-terminus) and/or the 5′ are presented to the left and the carboxyl group (C-terminus) and/or 3′ are presented to the right.

As used herein, the term “peptide” refers to a molecular chain of amino acids, which, if required, can be modified in vivo or in vitro, for example by manosylation, glycosylation, amidation (specifically C-terminal amides), carboxylation or phosphorylation with the stipulation that these modifications must preserve the biological activity of the original molecule. In addition, peptides can be part of a chimeric protein.

Functional derivatives of the peptides are also included in the present invention. Functional derivatives are meant to include peptides which differ in one or more amino acids in the overall sequence, which have deletions, substitutions, inversions or additions. Amino acid substitutions which can be expected not to essentially alter biological and immunological activities have been described. Amino acid replacements between related amino acids or replacements which have occurred frequently in evolution include, inter alia Ser/Ala, Ser/Gly, Asp/Gly, Asp/Asn and Ile/Val.

The peptides according to the invention can be produced synthetically or by recombinant DNA technology. Methods for producing synthetic peptides are well known in the art.

The organic chemical methods for peptide synthesis are considered to include the coupling of the required amino acids by means of a condensation reaction, either in homogenous phase or with the aid of a so-called solid phase. The condensation reaction can be carried out as follows: Condensation of a compound (amino acid, peptide) with a free carboxyl group and protected other reactive groups with a compound (amino acid, peptide) with a free amino group and protected other reactive groups, in the presence of a condensation agent. Condensation of a compound (amino acid, peptide) with an activated carboxyl group and free or protected other reaction groups with a compound (amino acid, peptide) with a free amino group and free or protected other reactive groups. Activation of the carboxyl group can take place, inter alia, by converting the carboxyl group to an acid halide, azide, anhydride, imidazolide or an activated ester, such as the N-hydroxy-succinimide, N-hydroxy-benzotriazole or p-nitrophenyl ester.

The most common methods for the above condensation reactions are: the carbodiimide method, the azide method, the mixed anhydride method and the method using activated esters, such as described in The Peptides, Analysis, Synthesis, Biology Vol. 1-3 (Ed. Gross, E. and Meienhofer, J.) 1979, 1980, 1981 (Academic Press, Inc.).

In some embodiments, one or more of the peptides have a C-terminal amide.

As used herein, the term “peptide vaccine” refers to a preparation composed of at least one peptide that improves immunity to a particular pathogen.

In some embodiments, the immunogenic composition comprises at least one peptide. In some embodiments, the immunogenic composition comprises one peptide. In some embodiments, the peptide is a signal peptide. In some embodiments, the peptide is a signal peptide from a coronavirus. In some embodiments, the coronavirus is an alpha coronavirus. In some embodiments, the coronavirus is 229E or NL63. In some embodiments, the coronavirus is a beta coronavirus. In some embodiments, the coronavirus is OC43, HKU1, MERS-CoV or SARS-CoV.

In some embodiments, the coronavirus is SARS-CoV-2.

The term “adjuvant” as used herein refers to any component of a pharmaceutical composition that is not the active agent.

In some embodiments, the adjuvant is selected from the group comprising an oil emulsion, a cytokine, an immunostimulating complex (ISCOM), a saponin-type auxiliary agent, Montanide ISA 51VG, liposomes, aluminum hydroxide (alum), bovine serum albumin (BSA), keyhole limpet hemocyanin (KLH), Lipopolysacharrudes (LPS) or derivatives such as Monophosphoryl lipid A (MPL), CpG DNA, microbial DNA/RNA, nanoparticle (e.g., gold particles), bacterial ghosts, ligands or agonist antibodies for TNFa, TLR (Toll-like receptor)-based adjuvants (e.g. see Heit at al Eur. J. Immunol. (2007) 37:2063-2074) or a combination thereof.

In some embodiments, peptide antigens are associated with liposomes, such as lecithin liposomes or other liposomes known in the art.

In some embodiments, no adjuvant is added. In some embodiments, one adjuvant is added. In some embodiments, a combination of adjuvants is added.

In some embodiments, the immunogenic composition is configured for co-administration with one or more anti-infective agents.

In some embodiments, the peptide is preloaded in an isolated antigen-presenting cell. In some embodiments, the antigen-presenting cell comprises a dendritic cell, a macrophage, a B cell or any nucleated cell type present in the human body. Each possibility represents a separate embodiment.

### Methods of Treatment or Prevention

In some embodiments, the present invention is a vaccine comprising the immunogenic composition. In some embodiments, the present invention provides for signal peptide-based vaccine candidates against intracellular pathogens. In some embodiments, the vaccine is a preventive vaccine. In some embodiments, the vaccine is a therapeutic vaccine.

In some embodiments, the present invention is directed to a method of treating or preventing a pathogenic infection comprising administering the immunogenic composition comprising at least one peptide selected from the group SEQ ID NOs: 1-6 to a subject in need thereof.

In some embodiments, the pathogenic infection is a viral infection. In some embodiments, the viral infection is a coronavirus infection. In some embodiments, the coronavirus infection is caused by 229E (alpha coronavirus), NL63 (alpha coronavirus), OC43 (beta coronavirus) or HKU1 (beta coronavirus). In some embodiments, the coronavirus is Middle East Respiratory Syndrome (MERS) or Severe Acute Respiratory Syndrome (SARS). In some embodiments, the coronavirus infection is COVID-19.

The term “subject” as used herein refers to an animal, more particularly to non-human mammals and human organism. Non-human animal subjects may also include prenatal forms of animals, such as, e.g., embryos or fetuses. Non-limiting examples of non-human animals include: horse, cow, camel, goat, sheep, dog, cat, non-human primate, mouse, rat, rabbit, hamster, guinea pig, pig. In some embodiments, the subject is a human. Human subjects may also include fetuses.

As used herein, the terms “subject,” refers to any subject, particularly a mammalian subject, for whom therapy is desired, for example, a human.

In some embodiments, a subject in need thereof is afflicted with a pathogenic infection. In some embodiments, a subject in need thereof is susceptible to a pathogenic infection. In some embodiments, a subject in need thereof is potentially susceptible to a pathogenic infection.

The term “treating” as used herein encompasses alleviation of at least one symptom thereof, a reduction in the severity thereof, or inhibition of the progression thereof. Treatment need not mean that the disease, disorder, or condition is totally cured. To be an effective treatment, a useful composition herein needs only to reduce the severity of a disease, disorder, or condition, reduce the severity of symptoms associated therewith, or provide improvement to a patient or subject's quality of life.

In some embodiments, the peptide vaccine of the invention reduces transfection or transmission to other subjects,

In some embodiments, the peptide vaccine of the invention is administered in an immunogenically effective amount with or without a co-stimulatory molecule, agent or adjuvant. According to the method of the invention, the peptide vaccine may be administrated to a subject in need of such treatment for a time and under conditions sufficient to prevent, and/or ameliorate the pathogen infection.

In some embodiments, the immunogenic composition of the invention may be used in conjunction with a co-stimulatory molecule. Both molecules may be formulated separately or as a “chimeric vaccine” formulation, with a pharmaceutically acceptable carrier and administered in an amount sufficient to elicit a T lymphocyte-mediated immune response and/or a humoral response.

In some embodiments, the immunogenic composition may be administered to subjects by a variety of administration modes, including by intradermal, intramuscular, subcutaneous, intravenous, intra-atrial, intra-articular, intraperitoneal, parenteral, oral, rectal, intranasal, intrapulmonary, and transdermal delivery, or topically to the eyes, ears, skin or mucous membranes. Alternatively, the antigen may be administered ex-vivo by direct exposure to cells, tissues or organs originating from a subject (autologous) or another subject (allogeneic), optionally in a biologically suitable, liquid or solid carrier.

In certain embodiments of the invention, the peptides or pharmaceutical compositions with or without a co-stimulatory molecule are delivered to a common or adjacent target site in the subject, for example to a specific target tissue or cell population in which the vaccine formulation is intended to elicit an immune response. Typically, when the peptide or pharmaceutical composition and the optional co-stimulatory molecule are administered separately, they are delivered to the same or closely proximate site(s), for example to a single target tissue or to adjacent sites that are structurally or fluidly connected with one another (e.g., to allow direct exposure of the same cells, e.g., fluid flow transfer, dissipation or diffusion through a fluid or extracellular matrix of both vaccine agents). Thus, a shared target site for delivery of antigen and co-stimulatory molecule can be a common surface (e.g., a mucosal, basal or lumenal surface) of a particular target tissue or cell population, or an extracellular space, lumen, cavity, or structure that borders, surrounds or infiltrates the target tissue or cell population.

For prophylactic and treatment purposes, the peptide vaccine with or without a co-stimulatory molecule may be administered to the subject separately or together, in a single bolus delivery, via continuous delivery (e.g., continuous intravenous or transdermal delivery) over an extended time period, or in a repeated administration protocol (e.g., on an hourly, daily or weekly basis). The various dosages and delivery protocols contemplated for administration of peptide and co-stimulatory molecule, in simultaneous or sequential combination, are immunogenically effective to prevent, inhibit the occurrence or alleviate one or more symptoms of infection in the subject. An “immunogenically effective amount” of the peptide thus refers to an amount that is, in combination, effective, at dosages and for periods of time necessary, to elicit a specific T lymphocyte mediated immune response and/or a humoral response. This response can be determined by conventional assays for T-cell activation, including but not limited to assays to detect antibody production, proliferation, specific cytokine activation and/or cytolytic activity, e.g., using an antibody concentration/titer assay (e.g. via ELISA).

For prophylactic and therapeutic use, peptide antigens might be formulated with a “pharmaceutical acceptable carrier”. As used herein, “pharmaceutically acceptable carrier” includes any and all solvents, dispersion media, coatings, antibacterial and antifungal agents, isotonic and absorption enhancing or delaying agents, and other excipients or additives that are physiologically compatible. In specific embodiments, the carrier is suitable for intranasal, intravenous, intramuscular, intradermal, subcutaneous, parenteral, oral, transmucosal or transdermal administration. Depending on the route of administration, the active compound may be coated in a material to protect the compound from the action of acids and other natural conditions which may inactivate the compound.

Peptide vaccines may be administered to the subject per se or in combination with an appropriate auxiliary agent or adjuvant via injection. Alternatively, the peptide vaccine may be percutaneously administered through mucous membrane by, for instance, spraying the solution. The unit dose of the peptide typically ranges from about 0.01 mg to 100 mg, more typically between about 100 micrograms to about 5 mg, which may be administered, one time or repeatedly, to a patient.

Examples of auxiliary agents or adjuvants which can be formulated with or conjugated to peptide or protein antigens and/or vectors for expressing co-stimulatory molecules to enhance their immunogenicity for use within the invention include cytokines (e.g. GM-CSF), bacterial cell components such as BCG bacterial cell components, immnunostimulating complex (ISCOM), extracted from the tree bark called QuillA, QS-21, a saponin-type auxiliary agent, Montanide ISA 51VG, liposomes, aluminum hydroxide (alum), bovine serum albumin (BSA), tetanus toxoid (TT), keyhole limpet hemocyanin (KLH), and TLR (Toll-like receptor)-based adjuvants (e.g. see Heit at al Eur. J. Immunol. (2007) 37:2063-2074).

In preparing pharmaceutical compositions of the present invention, it may be desirable to modify the peptide antigen, or to combine or conjugate the peptide with other agents, to alter pharmacokinetics and biodistribution. A number of methods for altering pharmacokinetics and biodistribution are known to persons of ordinary skill in the art. Examples of such methods include protection of the proteins, protein complexes and polynucleotides in vesicles composed of other proteins, lipids (for example, liposomes), carbohydrates, or synthetic polymers. For example, the vaccine agents of the invention can be incorporated into liposomes in order to enhance pharmacokinetics and biodistribution characteristics. A variety of methods are available for preparing liposomes, as described in, e.g., U.S. Pat. Nos. 4,235,871, 4,501,728 and 4,837,028. For use with liposome delivery vehicles, peptides are typically entrapped within the liposome, or lipid vesicle, or are bound to the outside of the vesicle.

In some embodiments, the present invention provides a method of treating or preventing a pathogenic infection comprising administering an enriched T cell population to a subject in need thereof, wherein the enriched T cell population is obtained by administering the immunogenic composition to a T cell population in vitro.

In some embodiments, the pathogenic infection is a viral infection. In some embodiments, the viral infection is a coronavirus infection. In some embodiments, the coronavirus infection is caused by 229E (alpha coronavirus), NL63 (alpha coronavirus), OC43 (beta coronavirus) or HKU1 (beta coronavirus). In some embodiments, the coronavirus is Middle East Respiratory Syndrome (MERS) or Severe Acute Respiratory Syndrome (SARS). In some embodiments, the coronavirus infection is COVID-19.

In some embodiments, the present invention provides a method for generating anti-coronavirus antibodies, comprising administering the immunogenic composition to an animal or human subject.

As used herein, the term “antibody” refers to a polypeptide or group of polypeptides that include at least one binding domain that is formed from the folding of polypeptide chains having three-dimensional binding spaces with internal surface shapes and charge distributions complementary to the features of an antigenic determinant of an antigen. An antibody typically has a tetrameric form, comprising two identical pairs of polypeptide chains, each pair having one “light” and one “heavy” chain. The variable regions of each light/heavy chain pair form an antibody binding site. An antibody may be oligoclonal, polyclonal, monoclonal, chimeric, camelised, CDR-grafted, multi-specific, bi-specific, catalytic, humanized, fully human, anti-idiotypic and antibodies that can be labeled in soluble or bound form as well as fragments, including epitope-binding fragments, variants or derivatives thereof, either alone or in combination with other amino acid sequences. An antibody may be from any species. The term antibody also includes binding fragments, including, but not limited to Fv, Fab, Fab′, F(ab′)2, single stranded antibody (svFC), dimeric variable region (Diabody) and disulphide-linked variable region (dsFv). In particular, antibodies include immunoglobulin molecules and immunologically active fragments of immunoglobulin molecules, i.e., molecules that contain an antigen binding site. Antibody fragments may or may not be fused to another immunoglobulin domain including but not limited to, an Fc region or fragment thereof. The skilled artisan will further appreciate that other fusion products may be generated including but not limited to, scFv-Fc fusions, variable region (e.g., VL and VH)˜Fc fusions and scFv-scFv-Fc fusions.

Immunoglobulin molecules can be of any type (e.g., IgG, IgE, IgM, IgD, IgA and IgY), class (e.g., IgG1, IgG2, IgG3, IgG4, IgA1 and IgA2) or subclass.

In some embodiments, the animal is a horse, cow, camel, goat, sheep, dog, cat, non-human primate, mouse, rat, rabbit, hamster, guinea pig or pig. An animal may also include prenatal forms of animals, such as, e.g., embryos or fetuses.

In some embodiments, the anti-coronavirus antibodies are antibodies against alpha coronavirus peptides. In some embodiments, the coronavirus is 229E or NL63. In some embodiments, the anti-coronavirus antibodies are antibodies against beta coronavirus peptides. In some embodiments, the coronavirus is OC43, HKU1, MERS-CoV or SARS-CoV.

In some embodiments, the anti-coronavirus antibodies are antibodies against SARS-CoV-2 peptides. In some embodiments, the anti-coronavirus antibodies are antibodies against SARS-CoV-2 signal peptides.

General

As used herein, the term “about” when combined with a value refers to plus and minus 10% of the reference value. For example, a length of about 1000 nanometers (nm) refers to a length of 1000 nm ±100 nm.

It is noted that as used herein and in the appended claims, the singular forms “a,” “an,” and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Various embodiments and aspects of the present invention as delineated hereinabove and as claimed in the claims section below find experimental support in the following examples.

EXAMPLES

Generally, the nomenclature used herein, and the laboratory procedures utilized in the present invention include molecular, biochemical, microbiological, and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells-A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Strategies for Protein Purification and Characterization-A Laboratory Course Manual” CSHL Press (1996); all of which are incorporated by reference. Other general references are provided throughout this document.

TABLE 1

| | Signa | | | | | | B- | Cum- |
|---|---|---|---|---|---|---|---|---|
| | IP-5.0 | | | | HLA- | HLA- | cell | ulative |
| | Sec/ | | Sequence\ | | I | II | epitope | score |
| Protein | SPI | length | (SEQ ID NO:) | Homology | (%) | (%) | (%) | (%) |
| Surface | 0.9739 | 15 | MFVFLVLLPL | No | 74.9 | 75.4 | 25.6 | 14.07 |
| glyco- | | | VSSQC (SEQ | | | | | |
| protein | | | ID NO: 1) | | | | | |
| ORF7a | 0.9951 | 15 | MKIILFLALIT | No | 78.1 | 83.5 | 25.3 | 16.39 |
| | | | LATC | | | | | |
| | | | (SEQ ID NO: 2) | | | | | |
| ORF8 | 0.997 | 15 | MKFLVFLGIIT | No | 65.6 | 57.7 | 20.9 | 7.89 |
| | | | TVAA (SEQ ID | | | | | |
| | | | NO: 3) | | | | | |

### EXAMPLE 1

### In Vivo Immunogenicity

BALB/c and C57BL/6 mice were immunized by subcutaneous (SC) injections with 100 μg of individual SPs or a SP mixture and with intraperitoneal injections of 100 ng of mouse granulocyte macrophage colony stimulating factor (mGM-CSF) three times at 7-day intervals.

To monitor if antibody response was developed against the SPs an enzyme-linked immunosorbent assay (ELISA) was performed with sera obtained on days 10 and 20 post the last immunization when VXL-301, or VXL-302, or VXL-303 or VXL-201c (negative control) were coated on the ELISA plate.

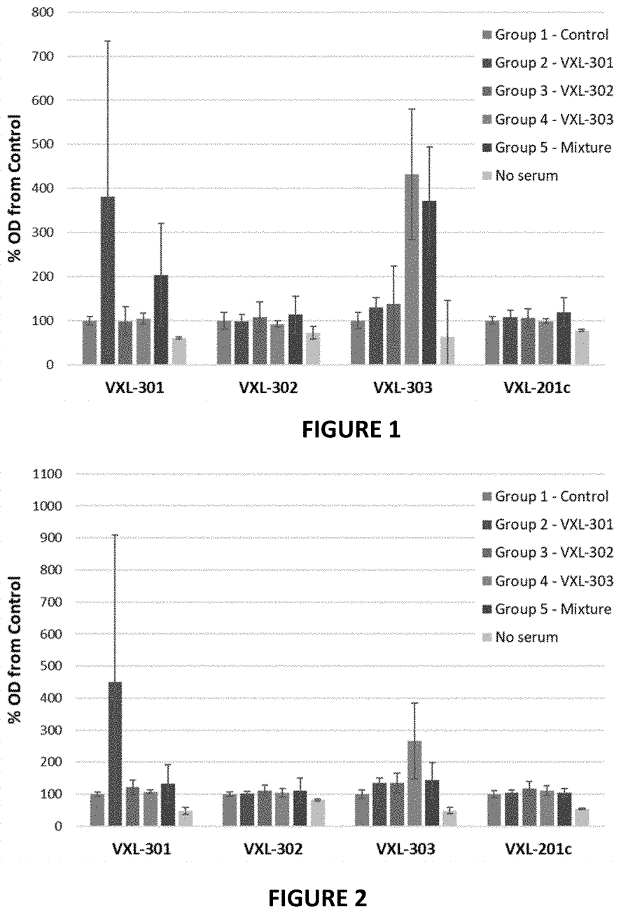

In the BALB/c mice (FIG. 1), the greatest antibody response was observed at day 10 against VXL-301 (SEQ ID NO: 1) for mice immunized with VXL-301 and for mice immunized with the mixture of VXL-301+VXL-302+VXL-303 (SEQ ID Nos.: 1-3), and against VXL-303 (SEQ ID NO: 3) for mice immunized with VXL-303 and for mice immunized with the mixture of VXL-301+VXL-302+VXL-303 (SEQ ID Nos.: 1-3).

For the C57BL/6 mice the highest antibody responses were observed at day 20 against VXL-301 for mice immunized with VXL-301, and against VXL-303 for mice immunized with VXL-303 (FIG. 2).

The ELISA results demonstrated antibody responses for mice immunized with VXL-301, or with VXL-303, or with the mixture of VXL-301+VXL-302+VXL-303. Therefore, since humoral immune response was observed it was decided to continue with further studies.

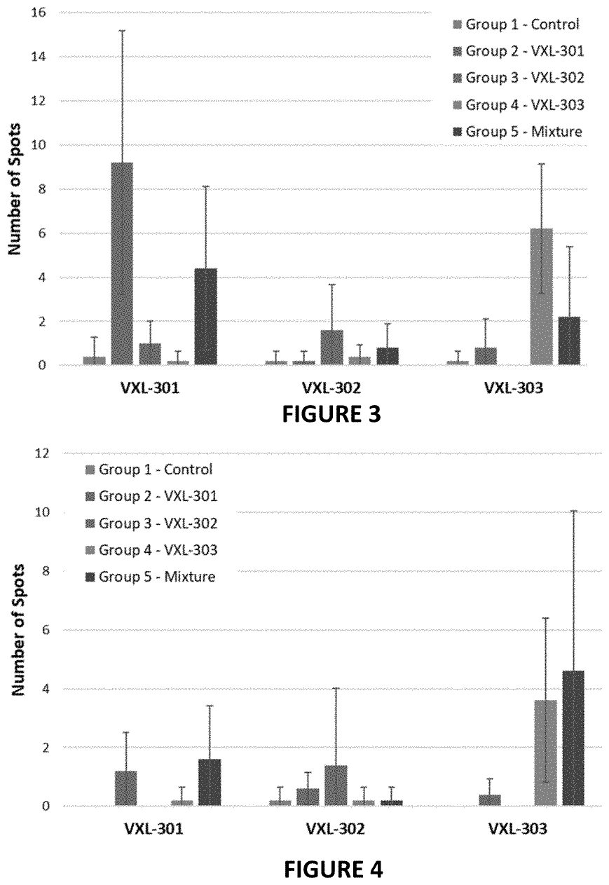

Additionally, an ELISpot SP stimulation experiment on splenocytes of immunized C57BL/6 mice showed that VXL-301 induced secretion of interleukin 2 primarily in the VXL-301- and the SP mixture-immunized groups. VXL-303 induced secretion of interleukin 2 primarily in the VXL-303- and the SP mixture-immunized groups. VXL-302 induced secretion of interleukin 2 primarily in the VXL-302-immunized group (FIG. 3). Similarly, an ELISpot SP stimulation experiment on splenocytes of immunized BALB/c mice showed that VXL-301 induced secretion of interferon gamma primarily in the VXL-301- and the SP mixture-immunized groups. VXL-303 induced secretion of interferon gamma primarily in the VXL-303- and the SP mixture-immunized groups. VXL-302 induced secretion of interferon gamma primarily in the VXL-302 immunized group (FIG. 4).

### EXAMPLE 2

### Ex Vivo Immunogenicity

Immunogenicity is assessed by quantifying proliferation of T cells in response to exposure to peptides. To this end, healthy donor peripheral blood mononuclear cells (PBMCs) are marked by a fluorescent dye and exposed to either the naked peptide or to autologous antigen presenting cells (APCs) pre-loaded with the SP. Phytohaemagglutinin (PHA), or tetanus toxoid, (TT), or previously tested SPs, serve as a positive control for maximum stimulation. Unloaded APCs, or medium, or human Fab fragments serve as negative controls. Proliferation is calculated by the increase of CD3+ T cells and the fraction of cells with different number of cell divisions assessed by flow cytometry.

Peripheral blood mononuclear cells (PBMCs) from healthy donors were stimulated by SP-loaded autologous dendritic cells (DCs). VXL-301, VXL-302, and VXL-303, as well as modified peptides (e.g., as provided under SEQ ID NO: 4-6) both individually and in combination, exhibited proliferation that was greater than unloaded DCs (non-specific proliferation). Proliferation was comparable to the levels observed for positive control SPs, and proliferation was also evident in both CD4+ cytotoxic T cells and CD8+ helper T cells.

### EXAMPLE 3

### Cytokine Secretion

Activation of T cells after immunogenic stimulation leads to cytokine secretion. Assessing the cytokine secretion level is used as an indication of T cell activation. The identity of the cytokine(s) can be used to determine whether the response is a Th1 or Th2 immune response, by assessing, e.g., IFN-γ and IL-2 (Th1) or IL-4 (Th2).

This Standard of Procedure (SOP) will describe quantification of cytokine secretion using ELISA. Other means of detection may include multiplex (e.g., Luminex) and ELISPOT.

Cell supernatant will be assessed for cytokine concentration by enzyme-linked immunosorbent assay (ELISA), where a 96-well plate is coated with the capture antibody. The sample will be added for the cytokine to be identified and isolated by this antibody. A second antibody (“detection antibody”) that detect the cytokine will be added to “sandwich” the cytokine. This second cytokine is coupled to biotin. Horseradish peroxidase (HRP)-conjugated streptavidin binds the biotin. HRP will react with its substrate, TMB/E, to produce a product with a color. The color will be measured with a specific wavelength as a measurement of the quantity of that product (colorimetric measurement). The amount of product correlated to the amount of secondary antibody and thus to the amount of cytokine.

### EXAMPLE 4

### Functional Test-Cytotoxicity

Activity of T cell clones will be measured by the ability of the T cell clones to lyse quasi-infected cells. T cell clones will be generated from healthy donor PBMCs by sequential exposure to the peptide. Anti-COVID-19 T cell clones will be co-cultured with autologous macrophages pre-loaded with the peptide (representing quasi-infected cells). Cytotoxicity will be assayed by pre-labelling macrophages with fluorescent dye, or by lactic dehydrogenase (LDH) release, or by propidium iodide (PI)/AnnexinV staining.

Although the invention has been described in conjunction with specific embodiments thereof, it is evident that many alternatives, modifications, and variations will be apparent to those skilled in the art. Accordingly, it is intended to embrace all such alternatives, modifications and variations that fall within the spirit and broad scope of the appended claims.

1. An immunogenic composition, comprising:
a) at least one peptide selected from the group consisting of: MFVFLVLLPLVSSQC (SEQ ID NO: 1), MKFLVFLGIITTVAA (SEQ ID NO: 3) and MKIILFLALITLATC (SEQ ID NO: 2), or a homolog thereof; and
b) optionally at least one adjuvant.

2. The immunogenic composition according to claim 1, wherein said at least one peptide has an amino acid sequence having at least 85% homology with said amino acid sequence selected from SEQ ID NOs: 1-3.

3. The immunogenic composition according to claim 1, wherein said at least one peptide is a peptide consisting of an amino acid sequence selected from the group selected from SEQ ID Nos.: 1-3.

4. The immunogenic composition according to claim 1, wherein the peptide comprises the amino acid sequence as set forth in SEQ ID NO: 1.

5. The immunogenic composition according to claim 1, wherein the peptide comprises the amino acid sequence as set forth in SEQ ID NO: 3.

6. The immunogenic composition according to claim 1, comprising SEQ ID NO: 1 and SEQ ID NO: 3.

7. The immunogenic composition according to claim 1, comprising SEQ ID NO: 1, SEQ ID NO: 3, and SEQ ID NO: 2.

8. The immunogenic composition according to claim 1, xhibiting, upon administration, a combined activation of both CD4+ and CD8+ T cells.

9. The immunogenic composition according to claim 1, being formulated for co-administration with one or more anti-infective agents.

10. The immunogenic composition according to claim 1, wherein one or more of said at least one peptide has a C-terminal amide.

11. The immunogenic composition according to claim 1, wherein one or more of said at least one peptide further comprises at least one lysine residue located at any one of the C-terminus and N-terminus of the peptide.

12. A vaccine comprising the immunogenic composition of claim 1.

13. An isolated antigen-presenting cell preloaded with at least one peptide selected from the group consisting of: MFVFLVLLPLVSSQC (SEQ ID NO: 1), MKFLVFLGIITTVAA (SEQ ID NO: 3) and MKIILFLALITLATC (SEQ ID NO: 2), or a homolog thereof.

14. A method for treating or preventing a pathogenic infection in a subject in need thereof, comprising administering to the subject a therapeutically effective amount of the immunogenic composition according to claim 1, thereby treating or preventing a pathogenic infection in the subject.

15. A method for treating or preventing a pathogenic infection in a subject in need thereof, comprising administering to the subject a therapeutically effective amount of an enriched T cell population, wherein said enriched T cell population is obtained by administration of the immunogenic composition according to claim 1 to a T cell population in vitro.

16. The method of claim 14, wherein said pathogenic infection is induced by a coronavirus.

17. The method of claim 16, wherein said coronavirus is the severe acute respiratory syndrome coronavirus 2 (SARS-CoV-2).

18. A method for generating anti-coronavirus antibodies comprising administering the immunogenic composition according to claim 1 to an animal or a human subject.

19. The method of claim 18, wherein said anti-coronavirus antibodies are characterized by having binding affinity to a signal peptide or a plurality thereof being derived from SARS-CoV-2.
